(19) United States
(12) Patent Application Publication
(10) Pub. No.: US 2003/0130203 A1
(43) Pub. Date: Jul. 10, 2003

(54) Medicament for combating respiratory depression

(76) Inventors: Thomas Christoph, Aachen (DE); Boris Chizh, Cambridge (GB); Oswald Zimmer, Wuerselen (DE)

Correspondence Address: PERMAN & GREEN, 425 POST ROAD, FAIRFIELD, CT, 06824, US

(21) Appl. No.: 10/276,955

(22) PCT Filed: May 5, 2001
(86) PCT No.: PCT/EP01/05110

(30) Foreign Application Priority Data
May 26, 2000 (DE) 10025949.9

(51) Int. Cl. A61K 38/04; A61K 9/14

(52) U.S. Cl. 514/19; 424/486; 424/488

(57) ABSTRACT

The invention relates to the use of at least one compound of general formula (I) and/or one of its diastereomers and/or one of its enantiomers and/or one of the corresponding physiologically compatible salts for producing a medicament for combating respiratory depression, with the exception of medicaments for combating respiratory depression as a cause of sleep apnea.

The present invention relates to the use of at least one compound of general formula I and/or one of its diastereoisomers and/or one of its enantiomers and/or one of the corresponding physiologically acceptable salts for the preparation of a medicament for combating respiratory depression, with the exception of medicaments for combating respiratory depression as a cause of sleep apnoea.

The occurrence of respiratory depression, e.g. when administering compounds with opioid activity, in states of shock, when administering psychotropic drugs or in cases of central respiratory regulation disorders, is a situation which arises relatively frequently in clinical practice and is not uncommonly life-threatening for the patient. There is therefore a worldwide need for effective therapies for combating respiratory depression, as documented in the large number of scientific papers which have recently appeared in this field from the sectors of both clinical practice and fundamental research.

The object of the invention was therefore to provide medicaments suitable for combating respiratory depression, especially for combating respiratory depression when administering compounds with opioid activity, in states of shock, when administering psychotropic drugs or in cases of central respiratory regulation disorders.

It has now been found, surprisingly, that the compounds of general formula I, their enantiomers, their diastereoisomers and the corresponding physiologically acceptable salts are suitable for combating respiratory depression, especially for combating respiratory depression when administering compounds with opioid activity, in states of shock, when administering psychotropic drugs or in cases of central respiratory regulation disorders.

The present invention therefore provides the use of at least one compound of general formula I:

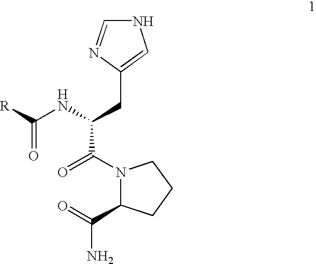

in which the radical R is one of the following groups a) to f):

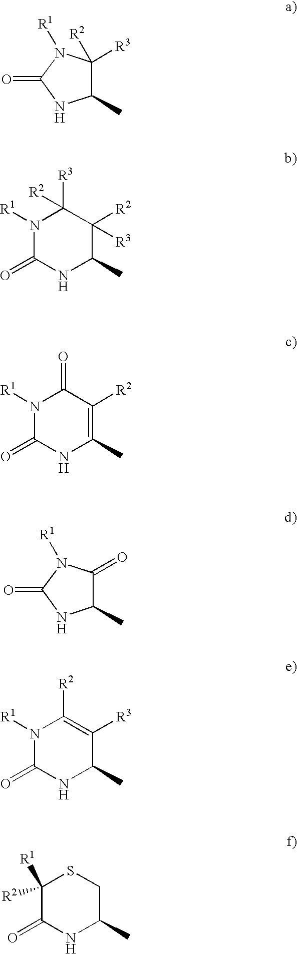

and the radicals R¹, R² and R³, which are identical or different, are an H or a CH₃ radical, and/or at least one of its enantiomers and/or one of its diastereoisomers and/or at least one corresponding physiologically acceptable salt, for the preparation of a medicament for combating respiratory depression, preferably for the preparation of a medicament for combating respiratory depression when administering compounds with opioid activity and/or in states of shock and/or when administering psychotropic drugs and/or in cases of central respiratory regulation disorders, with the exception of medicaments for combating respiratory depression as a cause of sleep apnoea.

The compounds of general formula I and their enantiomers and diastereoisomers and the corresponding physiologically acceptable salts can be prepared as disclosed in DE-PS-2449167, EP-0 429 245 or J. Med. Chem., 1990, 33(8), pp 2130 et seq., by conversion of the corresponding carboxylic acids known to those skilled in the art.

According to the invention, the compounds of general formula I, their enantiomers and diastereoisomers and the corresponding physiologically acceptable salts can be used individually or in mixtures of at least two of these compounds for the preparation of a medicament for combating respiratory depression. It is preferable to use only one compound of general formula I, one of its enantiomers, one of its diastereoisomers or one of the corresponding physiologically acceptable salts for the preparation of the medicament.

In one preferred embodiment of the present invention, at least one compound of general formula I is used in which the radical R is the group f), R¹ is a CH₃ radical and R² is an H radical, and/or at least one of its enantiomers and/or one of its diastereoisomers and/or at least one of its physiologically compatible salts is used.

In another preferred embodiment of the present invention, a compound of general formula I is used in which the radical R is the group a) and the radicals R¹, R² and R³ are each H, and/or at least one of its enantiomers and/or one of its diastereoisomers and/or at least one of its corresponding physiologically acceptable salts is used.

It is also preferable to use a compound of general formula I in which the radical R is the group c), the radical R¹ is a CH₃ radical and the radical R² is an H radical, and/or at least one of its enantiomers and/or one of its diastereoisomers and/or at least one of its corresponding physiologically acceptable salts.

In one particularly preferred embodiment of the present invention, the compound of general formula I is used in which the radical R is the group f), R¹ is a CH₃ radical and R² is an H radical (montirelin), and/or at least one of its physiologically compatible salts is used.

As physiologically acceptable salts of the compounds of general formula I and/or their enantiomers and/or their diastereoisomers, it is preferable to use the hydrochloride, hydrobromide, sulphate, sulphonate, phosphate, tartrate, embonate, formate, acetate, propionate, benzoate, oxalate, succinate, citrate, glutamate, fumarate, aspartate, glutarate, stearate, butyrate, malonate, lactate, mesylate or a mixture of at least two of these salts.

The above-mentioned medicaments preferably take the form of tablets, chewing tablets, chewing gums, coated tablets (dragees), capsules, drops, juices, syrups, suppositories, solutions, emulsions, suspensions, powders or sprays. Particularly preferably, the medicaments take the form of tablets, capsules, drops or solutions.

The above-mentioned medicaments for combating respiratory depression are also preferably in multiparticulate form, formulated preferably as microtablets, microcapsules, spheroids, ion exchange resins, granules, active ingredient crystals or pellets and particularly preferably as microtablets, granules or pellets, optionally filled into capsules or compressed to tablets. In terms of the present invention, pellets also include those produced by extrusion and spheronization, or built-up pellets.

The medicaments are preferably suitable for oral, intravenous, intramuscular, subcutaneous, intrathecal, epidural, buccal, sublingual, pulmonary, rectal, transdermal, nasal or intracerebroventricular administration, medicaments for oral or intravenous administration being particularly preferred.

Preparations suitable for oral administration are preferably those in the form of tablets, chewing tablets, chewing gums, coated tablets (dragees), capsules, granules, drops, juices and syrups. A form suitable for buccal administration is preferably a transmucosal therapeutic system. Forms suitable for parenteral, topical and inhalational administration are preferably solutions, suspensions, emulsions, readily reconstitutable dry preparations, microspheroids, sprays, suppositories or plasters (e.g. transdermal therapeutic systems). Particularly preferred forms are suppositories or solutions for parenteral administration, transdermal therapeutic systems for topical administration and inhaling solutions or powders for inhalational administration.

In addition to at least one compound of general formula I and/or one of its enantiomers and/or one of its diastereoisomers and/or at least one of the corresponding physiologically acceptable salts, the medicaments can preferably be formulated using excipients, fillers, solvents, diluents, colours, flavourings, binders or mixtures of at least two of these materials. The choice of these adjuncts and their amounts depends on the manner in which the medicament is to be administered. Those skilled in the art are familiar with the adjuncts suitable for each particular dosage form, and their amounts. The medicaments can be prepared by the conventional methods known to those skilled in the art.

The above-mentioned medicaments for combating respiratory depression can also contain a sustained-release form of at least one compound of general formula I, one of its enantiomers, one of its diastereoisomers and/or one of the corresponding physiologically acceptable salts.

Retardation of the particular active ingredient is preferably effected by applying a sustained-release coating, by binding to an ion exchange resin, by embedding in a sustained-release matrix or by a combination of these measures.

Suitable sustained-release coatings include water-insoluble waxes or polymers, e.g. acrylic resins, preferably poly(meth)acrylates, or water-insoluble celluloses, preferably ethyl cellulose. These materials are known from the state of the art, e.g. Bauer, Lehmann, Osterwald, Rothgang, “Überzogene Arzneiformen” (“Coated dosage forms”), Wissenschaftliche Verlagsgesellschaft mbH Stuttgart, 1988, pp 69 et seq. They are introduced here by way of reference and are thereby regarded as part of the disclosure.

In addition to the water-insoluble polymers, the sustained-release coatings can optionally also contain non-retarding, preferably water-soluble polymers in amounts of up to 30% by weight, such as polyvinylpyrrolidone, or water-soluble celluloses, preferably hydroxypropyl methyl cellulose or hydroxypropyl cellulose, and/or hydrophilic pore-forming agents such as sucrose, sodium chloride or mannitol, and/or the known plasticizers, in order to adjust the release rate of the particular active ingredient.

Moreover, the particular medicament formulation can optionally have further coatings. Other coatings which can be present are those which dissolve as a function of pH. Thus it is possible to formulate a medicament which passes through the stomach undissolved, the particular active ingredient only being released in the intestinal tract. It is also possible to use coatings which act as taste improvers.

Another conventional retardation procedure is to bind the particular active ingredient to ion exchange resins. These active ingredients are retarded using cation exchange resins, preferably polystyrenesulphonates.

The particular active ingredient can also be retarded in a sustained-release matrix, preferably as a uniform distribution. Matrix materials which can be used are physiologically acceptable hydrophilic materials known to those skilled in the art. The hydrophilic matrix materials used are preferably polymers and particularly preferably cellulose ethers, cellulose esters and/or acrylic resins. The matrix materials used are very particularly preferably ethyl cellulose, hydroxypropyl methyl cellulose, hydroxypropyl cellulose, hydroxymethyl cellulose, poly(meth)acrylic acid and/or derivatives thereof such as its salts, amides or esters.

Other preferred matrix materials are those consisting of hydrophobic materials such as hydrophobic polymers, waxes, fats, long-chain fatty acids, fatty alcohols or corresponding esters or ethers, or mixtures thereof. The hydrophobic materials used are particularly preferably C₁₂-C₃₀ fatty acid mono- or diglycerides and/or C₁₂-C₃₀ fatty alcohols and/or waxes or mixtures thereof.

Mixtures of said hydrophilic and hydrophobic materials can also be used as the sustained-release matrix material.

In another preferred embodiment of the present invention, the above-mentioned medicament contains at least one compound of general formula I and/or one of its enantiomers and/or one of its diastereoisomers and/or one of the corresponding physiologically acceptable salts in the non-retarded form as well as the sustained-release form. Combination with the immediately released active ingredient makes it possible to achieve a high initial dose for immediate combating of the respiratory depression. The slow release from the sustained-release form then prevents the respiratory depression from recurring.

The amount of active ingredient to be administered to the patient varies e.g. as a function of the patient's weight, the type of administration, the indication and the degree of severity of the respiratory depression.

The amount to be administered and the release of the particular active ingredient(s) are preferably adjusted so that the medicament needs to be administered at most twice and preferably only once a day.

If the medicament is administered once a day, the patient receives preferably 0.1 to 100 mg and particularly preferably 0.5 to 50 mg of a compound of general formula I and/or one of its diastereoisomers and/or one of its enantiomers and/or one of the corresponding physiologically acceptable salts.

If the medicament is administered twice a day, the patient receives preferably 0.05 to 50 mg and particularly preferably 0.25 to 25 mg of a compound of general formula I and/or one of its diastereoisomers and/or one of its enantiomers and/or one of the corresponding physiologically acceptable salts.

Surprisingly, the compounds of general formula I, their enantiomers, their diastereoisomers and the corresponding physiologically acceptable salts are found to be very effective in the combating of respiratory depression, especially in the combating of respiratory depression when administering compounds with opioid activity and/or in states of shock and/or when administering psychotropic agents and/or in cases of central respiratory regulation disorders.

Pharmacological Tests

Measurement of Respiratory Rate on the Awake Rat

To study the respiratory rate, awake male Sprague Dawley rats (Janvier, France) weighing 190 to 315 g were immobilized in Plexiglas tubes. For intravenous administration of the medicament solutions, the rats were each provided with a catheter in the caudal vein. The respiratory rate of the rats was measured via a water-filled balloon catheter located laterally between the rat and the Plexiglas tube. The balloon catheter was connected to a pressure sensor and a high-speed chart recorder (Gould, Dietzenbach).

After an equilibration period of 30 minutes, a baseline value for the respiratory rate was determined. The appropriate active ingredient solution was then administered intravenously and the respiratory rate was measured immediately and after 1, 2, 5, 10 and 15 minutes. Each rat received a single administration per day.

The invention is illustrated below with the aid of Examples. These Examples serve to illustrate the invention without restricting the general inventive idea.

### EXAMPLE

### Example 1

To study the effect on respiratory depression when administering compounds with opioid activity, each of a group of 8 rats received intravenously a 0.9% saline solution containing 10 mg of morphine per kg of body weight and 0.215 mg of the compound of general formula I in which the radical R is the group f), the radical R¹ is CH₃ and the radical R² is H (montirelin) per kg of body weight.

### Example 2

To study the effect on respiratory depression when administering compounds with opioid activity, each of a second group of 8 rats received intravenously a 0.9% saline solution containing 46.4 mg of the compound of general formula I in which the radical R is the group c), the radical R¹ is a CH₃ radical and the radical R² is H per kg of body weight and 10 mg of morphine per kg of body weight.

Comparative Example 1:

For comparison, each of a third group of 8 rats received intravenously a 0.9% saline solution containing only 10 mg of morphine per kg of body weight.

Comparative Example 2:

For comparison, each of a fourth group of 8 rats received intravenously a 0.9% saline solution in a volume of 1 ml per kg of body weight.

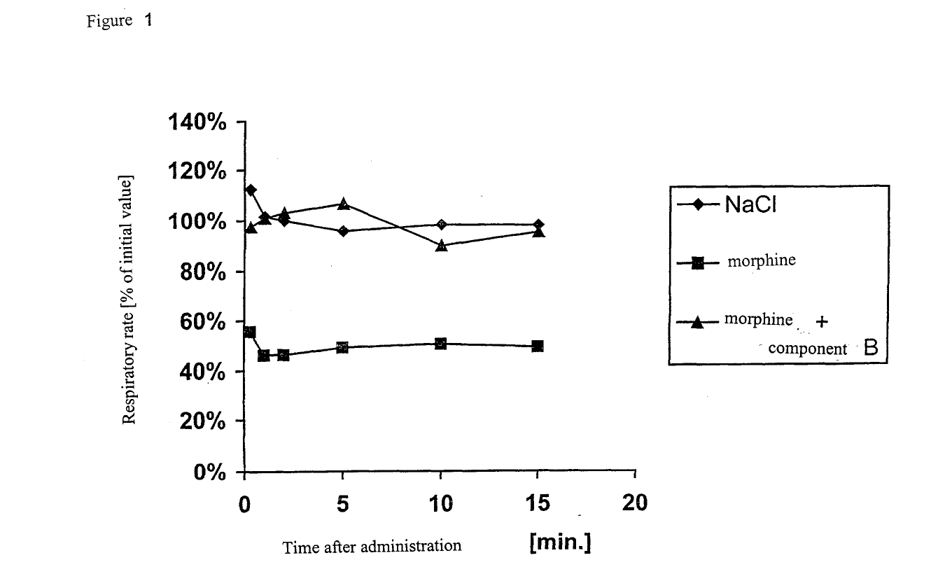

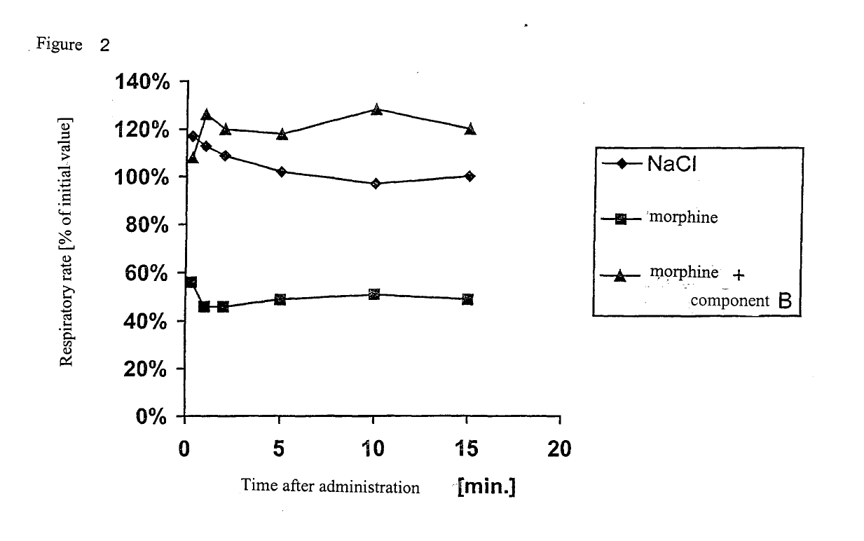

The results of these tests are shown in FIGS. 1 and 2.

As can be seen from FIGS. 1 and 2, the administration of a solution according to Comparative Example 1, containing morphine only, causes a marked decrease in respiratory rate.

By contrast, the administration of a solution according to Examples 1 and 2, containing in each case a compound of general formula I together with morphine, causes no decrease in respiratory rate; on the contrary, after administration of these solutions, the respiratory rate corresponds to the normal value for rats, as can also be observed when administering a solution according to Comparative Example 2, or is very slightly increased compared with this normal respiratory rate.

1. Use of at least one compound of general formula I: 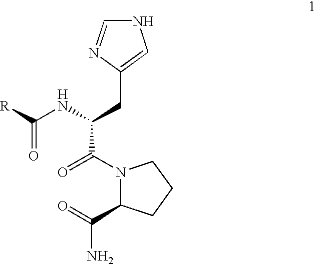
in which the radical R is one of the following groups a) to f): 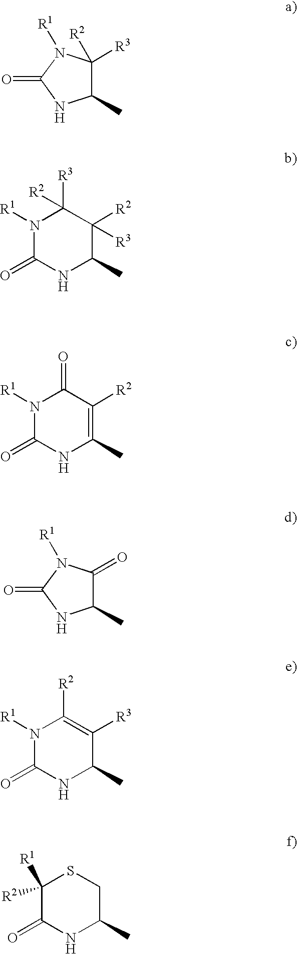
and the radicals R¹, R² and R³, which are identical or different, are an H or a CH₃ radical, and/or at least one of its enantiomers and/or at least one of its diastereoisomers and/or at least one corresponding physiologically acceptable salt, for the preparation of a medicament for combating respiratory depression, with the exception of medicaments for combating respiratory depression as a cause of sleep apnoea.

2. Use according to claim 1, characterized in that at least one compound of general formula I in which the radical R is the group f), R¹ is a CH₃ radical and R² is an H radical, and/or one of its enantiomers and/or one of its diastercoisomers and/or at least one corresponding physiologically acceptable salt is used.

3. Use according to claim 2, characterized in that the compound of general formula I in which the radical R is the group f), R¹ is a CH₃ radical and R² is an H radical, and/or at least one of its physiologically acceptable salts is used.

4. Use according to claim 1, characterized in that at least one compound of general formula I in which the radical R is the group a) and the radicals R¹, R² and R³ are each H, and/or one of its enantiomers and/or one of its diastereoisomers and/or at least one corresponding physiologically acceptable salt is used.

5. Use according to claim 1, characterized in that at least one compound of general formula I in which the radical R is the group c), the radical R¹ is a CH₃ radical and the radical R² is H, and/or one of its enantiomers and/or one of its diastereoisomers and/or at least one corresponding physiologically acceptable salt is used.

6. Use according to one of claims 1 to 5, characterized in that the physio-logically acceptable salt used is the hydrochloride, hydrobromide, sulphate, sulphonate, phosphate, tartrate, formate, acetate, propionate, benzoate, oxalate, succinate, citrate, glutamate, embonate, fumarate, aspartate, glutarate, stearate, butyrate, malonate, lactate, mesylate or a mixture of at least two of these salts.

7. Use according to one of claims 1 to 6 for combating respiratory depression when administering compounds with opioid activity, preferably morphine or fentanyl.

8. Use according to one of claims 1 to 6 for combating respiratory depression in states of shock.

9. Use according to one of claims 1 to 6 for combating respiratory depression when administering psychotropic drugs, preferably benzodiazepines.

10. Use according to one of claims 1 to 6 for combating respiratory depression in cases of central respiratory regulation disorders.

11. Use according to one of claims 1 to 10, characterized in that the medicament is in the form of tablets, chewing tablets, chewing gums, coated tablets (dragees), capsules, suppositories, transmucosal therapeutic systems, transdermal therapeutic systems or drops, or in the form of a juice, syrup, solution, emulsion, suspension, powder, readily reconstitutable dry preparation or spray, preferably in the form of tablets, capsules, drops or a solution.

12. Use according to one of claims 1 to 10, characterized in that the medicament is in multiparticulate form, preferably in the form of microtablets, microcapsules, spheroids, ion exchange resins, granules, active ingredient crystals or pellets and particularly preferably in the form of microtablets, granules or pellets, optionally filled into capsules or compressed to tablets.

13. Use according to one of claims 1 to 12, characterized in that the medicament is suitable for oral, intravenous, intramuscular, subcutaneous, intrathecal, epidural, buccal, sublingual, rectal, pulmonary, transdermal, nasal or intracerebroventricular administration, preferably for oral or intravenous administration.

14. Use according to one of claims 1 to 13, characterized in that at least one compound of general formula I is present in sustained-release form.

15. Use according to claim 14, characterized in that the retardation of the particular active ingredient is effected by applying a sustained-release coating, by binding to an ion exchange resin, by embedding in a sustained-release matrix or by a combination of these measures.

16. Use according to claim 15, characterized in that the coating is based on a water-insoluble polymer or wax.

17. Use according to claim 16, characterized in that the water-insoluble polymer used is a polyacrylic resin or a cellulose derivative, preferably an alkyl cellulose.

18. Use according to claim 17, characterized in that the polymer used is ethyl cellulose and/or a poly(meth)acrylate.

19. Use according to claim 15, characterized in that the matrix contains hydrophilic matrix material, preferably polymers, particularly preferably cellulose ethers, cellulose esters and/or acrylic resins and very particularly preferably ethyl cellulose, hydroxypropyl methyl cellulose, hydroxypropyl cellulose, hydroxymethyl cellulose, poly(meth)acrylic acid and/or its salts, amides and/or esters.

20. Use according to claim 15 or 19, characterized in that the matrix contains hydrophobic matrix material, preferably polymers, waxes, fats, long-chain fatty acids, fatty alcohols or corresponding esters or ethers, or mixtures thereof, and particularly preferably C₁₂-C₃₀ fatty acid mono- or diglycerides and/or C₁₂-C₃₀ fatty alcohols and/or waxes or mixtures thereof.

21. Use according to one of claims 14 to 20, characterized in that at least one compound of general formula I is present in a non-retarded form as well as the sustained-release form.
